(19) United States
(12) Patent Application Publication
(10) Pub. No.: US 2024/0376314 A1
(43) Pub. Date: Nov. 14, 2024

(54) POLYDIMETHYLSILOXANE-CONTAINING MONOMER HAVING PHOSPHORYLCHOLINE GROUP AND HYDROXYL GROUP

(71) Applicant: NOF CORPORATION, Tokyo (JP)

(72) Inventors: Yoshiki TANAKA, Kawasaki-shi, Kanagawa (JP); Shu TAKASHIMA, Kawasaki-shi, Kanagawa (JP); Norio IWAKIRI, Kawasaki-shi, Kanagawa (JP)

(73) Assignee: NOF CORPORATION, Tokyo (JP)

(21) Appl. No.: 18/686,529

(22) PCT Filed: Aug. 31, 2022
(86) PCT No.: PCT/JP2022/032690

§ 371 Date: Feb. 26, 2024

(30) Foreign Application Priority Data
Aug. 31, 2021 (JP) 2021-141703

(51) Int. Cl. C08L 83/10; C08G 77/00; C08G 77/20; C08G 77/28; C08G 77/442; G02B 1/04

(52) U.S. Cl. CPC C08L 83/10; C08G 77/20; C08G 77/28; C08G 77/442; G02B 1/043; C08G 77/80

(57) ABSTRACT

An object is to provide a polydimethylsiloxane-containing monomer that exhibits satisfactory compatibility with a hydrophilic monomer and a hydrophilic polymer, in which a polymerization product obtained by polymerizing a composition including the polydimethylsiloxane-containing monomer, the hydrophilic monomer, and the hydrophilic polymer has an anti-lipid adhesion property. It has been recognized that a polydimethylsiloxane-containing monomer having a phosphorylcholine group and a hydroxy group can achieve the object, and thus the present disclosure has been completed.

### TECHNICAL FIELD

The present disclosure relates to a polydimethylsiloxane-containing monomer having a phosphorylcholine group and a hydroxy group, a method of producing the monomer, a composition including the monomer, a polymerization product obtained by polymerizing the composition, and an ophthalmic device including the polymerization product.

The present application claims priority from Japanese Patent Application No. 2021-141703, which is incorporated herein by reference.

### BACKGROUND ART

In order to maintain the health state of the cornea, a contact lens that is an ophthalmic device needs to receive the supply of oxygen from air and requires oxygen permeability. In recent years, a contact lens using a siloxane monomer as a raw material has been developed in order to improve the oxygen permeability.

A contact lens requires wettability in addition to the oxygen permeability. It is said that a contact lens having high wettability has satisfactory wearing sensation and can be worn comfortably for a long period of time. In order to improve the wettability of a contact lens, a hydrophilic monomer is generally incorporated into a raw material.

In order to produce a contact lens having both high oxygen permeability and high wettability, there has been used a method including using both the siloxane monomer and the hydrophilic monomer as raw materials. However, the siloxane monomer generally has high hydrophobicity and has poor compatibility with the hydrophilic monomer. Thus, phase separation is liable to occur, resulting in difficulty in producing a transparent contact lens. In addition, an excellent contact lens needs to satisfy a plurality of elements, such as suitable mechanical strength, in addition to high oxygen permeability and high wettability. In order to satisfy those elements simultaneously, various investigations have been made on kinds and blending ratios of the siloxane monomer and the hydrophilic monomer with a view to optimizing formulation.

However, also in the investigations on formulation, compatibility between the siloxane monomer and the hydrophilic monomer and the accompanying transparency of a contact lens are problems, and hence there is a demand for a siloxane monomer having such high compatibility as to allow the siloxane monomer to be mixed with various hydrophilic monomers.

In order to produce a contact lens having both high oxygen permeability and high wettability, there has been known a method including mixing a contact lens composition with a hydrophilic polymer.

In Patent Literature 1, a contact lens produced through use of a composition mixed with a hydrophilic polymer such as polyvinylpyrrolidone (PVP) in addition to the siloxane monomer and the hydrophilic monomer exhibits more satisfactory wearing sensation because of high wettability. Meanwhile, there is a problem in that the hydrophilic polymer also has poor compatibility with the siloxane monomer similarly to the hydrophilic monomer, and hence phase separation is liable to occur.

In order to solve those problems, a siloxane monomer subjected to hydrophilization has been developed. For example, in Patent Literatures 2 and 3, there is a disclosure of a contact lens produced from a polydimethylsiloxane-containing monomer having a hydrophilic group. A polyether group, a hydroxy group, an amide group, or the like is selected as the hydrophilic group. However, in order to achieve high compatibility with the hydrophilic monomer and the hydrophilic polymer, it is required to introduce a large amount of the hydrophilic group with respect to polydimethylsiloxane, and hence there are concerns about adverse influences on other elements required for an excellent contact lens.

### CITATION LIST

### Patent Literature

[PTL 1] U.S. Pat. No. 6,822,016 B2
[PTL 2] JP 62-29776 B2
[PTL 3] JP 62-29777 B2

### SUMMARY OF INVENTION

### Technical Problem

A contact lens using a siloxane monomer as a raw material exhibits high oxygen permeability. Meanwhile, one of the problems of such contact lens is that lipid adhesion is liable to occur due to the hydrophobicity of the siloxane monomer, and the lipid adhesion causes a decrease in wearing sensation. A method such as surface modification is used to suppress the lipid adhesion. For example, a production process for a contact lens includes plasma treatment in some cases. However, the plasma treatment requires a dedicated facility, and hence there is a demand for a simpler surface modification method from the viewpoint of cost.

In view of the foregoing, an object of the present disclosure is to provide a polydimethylsiloxane-containing monomer that exhibits satisfactory a compatibility with hydrophilic monomer and a hydrophilic polymer, in which a polymerization product obtained by polymerizing a composition including the polydimethylsiloxane-containing monomer, the hydrophilic monomer, and the hydrophilic polymer has an anti-lipid adhesion property.

### Solution to Problem

In the present disclosure, it has been recognized that a polydimethylsiloxane-containing monomer having a phosphorylcholine group and a hydroxy group represented by the following formula (1B) can achieve the object, and thus the present disclosure has been completed.

That is, the present disclosure is as described below.

1. A polydimethylsiloxane-containing monomer having a phosphorylcholine group and a hydroxy group represented by the formula (1B) (compound represented by the formula (1B)):

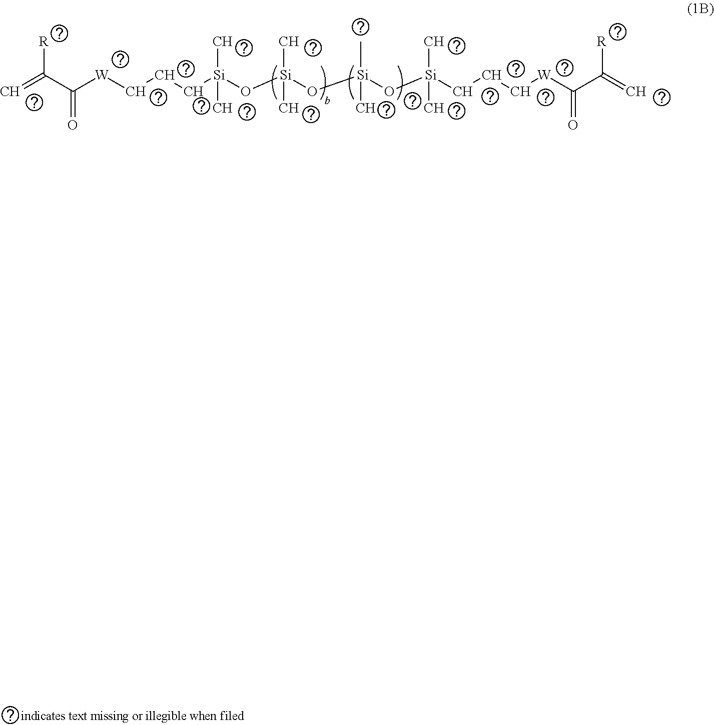

where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2);

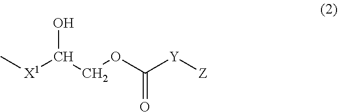

where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group;

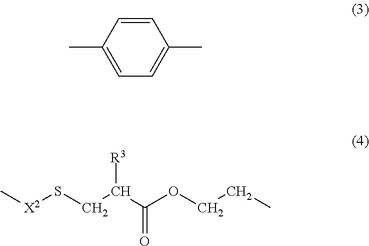

where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms.

2. A method of producing a polydimethylsiloxane-containing monomer having a phosphorylcholine group and a hydroxy group represented by the formula (1B), the method including a step of synthesizing the formula (1B) by: a hydrosilylation reaction between a compound represented by the formula (6B) and a vinyl compound having an epoxy group, and further a reaction between an epoxy group of a compound after the reaction and a carboxylic acid of a carboxylic acid compound having a phosphorylcholine group; or a reaction between an epoxy group of a vinyl compound having an epoxy group and a carboxylic acid of a carboxylic acid compound having a phosphorylcholine group, and further a hydrosilylation reaction between a vinyl compound having a phosphorylcholine group and a hydroxy group after the reaction and the compound represented by the formula (6B):

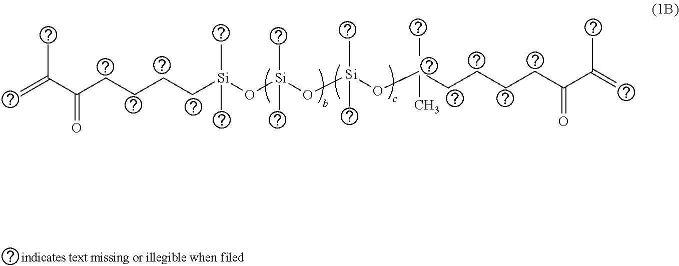

where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2);

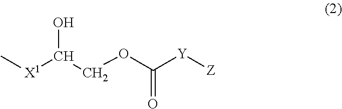

where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group;

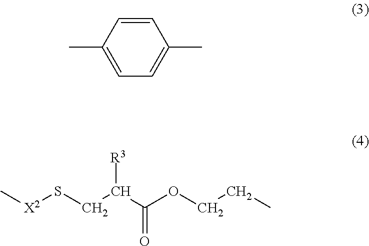

where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms;

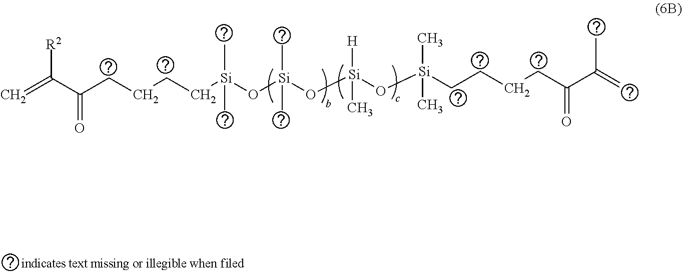

where R² represents H or CH₃, W² represents O or NR⁵, where R⁵ represents H or an alkyl group having 1 to 4 carbon atoms, “b” represents an integer of from 1 to 500, and “c” represents an integer of from 1 to 100.

3. A composition, including: a polydimethylsiloxane-containing monomer having a phosphorylcholine group and a hydroxy group represented by the following formula (1B); one or more kinds of hydrophilic monomers; and one or more kinds of hydrophilic polymers:

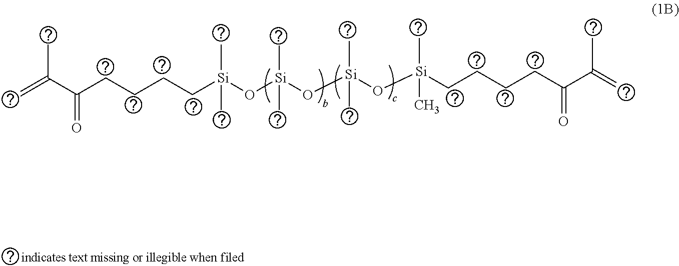

where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2);

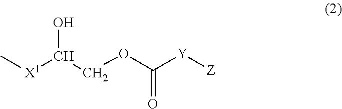

where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group;

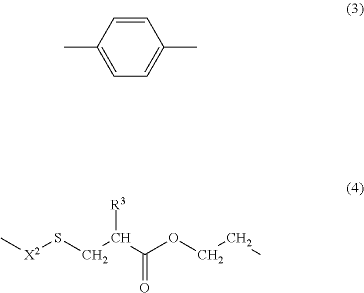

where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms.

4. A composition, including: a polydimethylsiloxane-containing monomer having a phosphorylcholine group and a hydroxy group represented by the following formula (1B); one or more kinds of hydrophilic monomers; one or more kinds of hydroxy group-containing siloxane monomers; and one or more kinds of hydrophilic polymers.

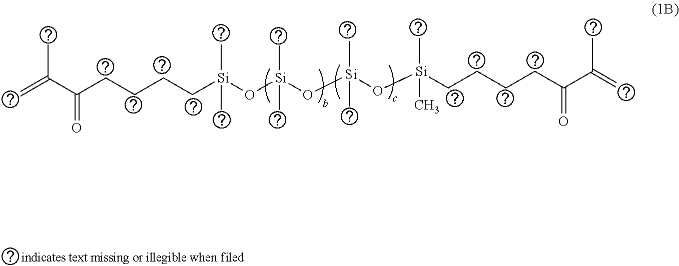

where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2);

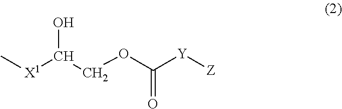

where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group;

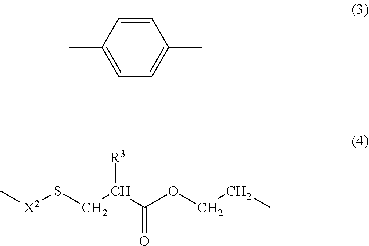

where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms.

5. The composition according to the above-mentioned item 3 or 4, wherein the hydrophilic polymer is one or more selected from the group consisting of: polyamide; polylactam; polyimide; polylactone; and polydextran.

6. The composition according to the above-mentioned item 3 or 4, wherein the hydrophilic polymer is polyvinylpyrrolidone.

7. A polymerization product obtained by polymerizing the composition of any one of the above-mentioned items 3 to 6.

8. An ophthalmic device, including the polymerization product of the above-mentioned item 7.

9. The compound represented by the formula (1B) according to the above-mentioned item 1, wherein the R² represents CH₃, the W² represents O, X¹ of the A⁰ represents CH₂CH₂CH₂—O—CH₂ or CH₂CH₂CH₂CH₂, and Y of the A⁰ is represented by the formula (3) or the formula (4) (where R³ represents CH₃ and X² represents CH₃) or represents CH₂.

10. The composition according to the above-mentioned item 3, wherein the R² represents CH₃, the W² represents O, X¹ of the A⁰ represents CH₂CH₂CH₂—O—CH₂ or CH₂CH₂CH₂CH₂, and Y of the A⁰ is represented by the formula (3) or the formula (4) (where R³ represents CH₃ and X² represents CH₃) or represents CH₂.

11. The composition according to the above-mentioned item 4, wherein the R² represents CH₃, the W² represents O, X¹ of the A⁰ represents CH₂CH₂CH₂—O—CH₂ or CH₂CH₂CH₂CH₂, and Y of the A⁰ is represented by the formula (3) or the formula (4) (where R³ represents CH₃ and X² represents CH₃) or represents CH₂.

12. A use of the composition of any one of the above-mentioned items 3 to 6 for producing an ophthalmic device.

### Advantageous Effects of Invention

The polydimethylsiloxane-containing monomer having a phosphorylcholine group and a hydroxy group of the present disclosure has satisfactory compatibility with a hydrophilic monomer and a hydrophilic polymer because of the presence of the phosphorylcholine group and the hydroxy group. A polymerization product obtained by polymerizing a composition including the polydimethylsiloxane-containing monomer, the hydrophilic monomer, and the hydrophilic polymer has transparency and an anti-lipid adhesion property.

### BRIEF DESCRIPTION OF DRAWINGS

FIG. 1 shows attribution results of ¹H NMR analysis of Example 1-1.

FIG. 2 shows attribution results of ¹H NMR analysis of Example 1-2.

### DESCRIPTION OF EMBODIMENTS

The present disclosure relates to a polydimethylsiloxane-containing monomer having a phosphorylcholine group and a hydroxy group. More specifically, the polydimethylsiloxane-containing monomer having a phosphorylcholine group and a hydroxy group of the present disclosure relates to a polydimethylsiloxane compound which is represented by the following formula (1B), contains a polydimethylsiloxane moiety, and further contains a phosphorylcholine group and a hydroxy group in a molecule thereof, and further a vinyl terminal group, and which is preferably polymerizable.

The polydimethylsiloxane-containing monomer having a phosphorylcholine group and a hydroxy group of the present disclosure exhibits satisfactory compatibility with a hydrophilic monomer and a hydrophilic polymer because of the presence of the phosphorylcholine group and the hydroxy group in a molecule thereof. When a composition of the polydimethylsiloxane-containing monomer, the hydrophilic monomer, and the hydrophilic polymer is polymerized, a transparent polymerization product is obtained. Further, the polymerization product exhibits an excellent anti-lipid adhesion property. In addition, when the polydimethylsiloxane-containing monomer has a vinyl terminal group, polymerization with any other composition (including monomers) becomes easy.

### <Polydimethylsiloxane-Containing Monomer Having Phosphorylcholine Group and Hydroxy Group of the Present Disclosure>

The polydimethylsiloxane-containing monomer having a phosphorylcholine group and a hydroxy group of the present disclosure is a polydimethylsiloxane-containing monomer represented by the formula (1B).

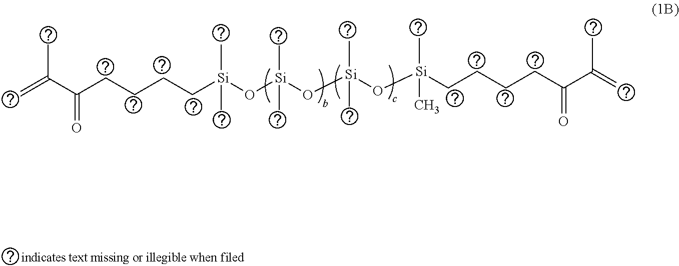

In the formula, R² represents H or CH₃, W² represents O or NR⁵, where R⁵ represents H or an alkyl group having 1 to 4 carbon atoms (e.g., a methyl group, an ethyl group, or a propyl group), “b” represents an integer of from 1 to 500, “c” represents an integer of from 1 to 100, and A⁰ is represented by the formula (2). In addition, repeating structures represented by “b” and “c” may include random sequences.

“b” and “c” are not particularly limited as long as “b” and “c” fall within the above-mentioned ranges. However, “b” represents from 1 to 500, preferably from 2 to 300, more preferably from 5 to 150, still more preferably from 10 to 100, particularly preferably from 15 to 75. “c” represents from 1 to 100, preferably from 1 to 70, more preferably from 1 to 30, still more preferably from 1 to 15, particularly preferably from 1 to 10, most preferably from 1 to 6.

In addition, the ranges (preferred ranges, more preferred ranges, and the like) of “b” and “c” may be combined or changed. For example, the preferred range may be selected for “b”, and the more preferred range may be selected for “c”.

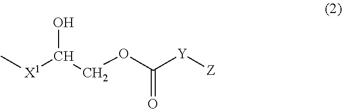

In the formula, X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group.

As used herein, the term “alkylene having 2 to 6 carbon atoms” refers to a divalent group obtained by removing two hydrogen atoms from an alkyl having 2 to 6 carbon atoms. The same also applies to other similar terms. An alkylene group forms two bonds with another group in an organic compound.

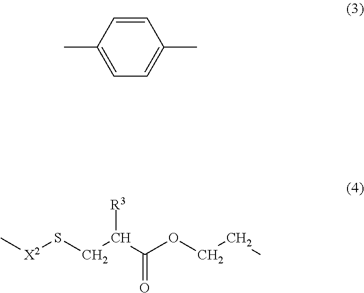

In the formula, R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms.

Z represents a phosphorylcholine group, and may be exemplified by the following formula (5).

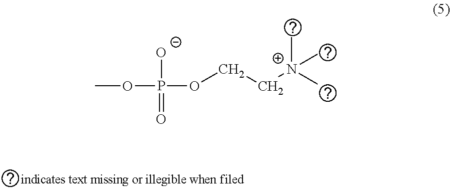

<Method of Producing Polydimethylsiloxane-Containing Monomer having Phosphorylcholine Group and Hydroxy Group of the Present Disclosure>

The polydimethylsiloxane-containing monomer having a phosphorylcholine group and a hydroxy group represented by the formula (1B) of the present disclosure is produced from a compound represented by the formula (6B) by combining a hydrosilylation reaction and a reaction between an epoxy group and a carboxylic acid.

More specifically, the reactions include: a hydrosilylation reaction between the compound represented by the formula (6B) and a vinyl compound having an epoxy group, and further a reaction between an epoxy group of a compound after the reaction and a carboxylic acid of a carboxylic acid compound having a phosphorylcholine group; or a reaction between an epoxy group of a vinyl compound having an epoxy group and a carboxylic acid of a carboxylic acid compound having a phosphorylcholine group, and further a hydrosilylation reaction between a vinyl compound having a phosphorylcholine group and a hydroxy group after the reaction and the compound represented by the formula (6B).

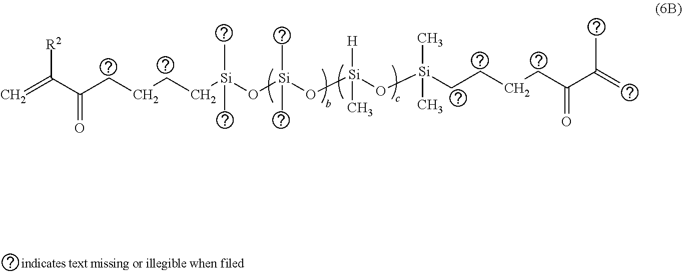

In the formula, R² represents H or CH₃, W² represents O or NR⁵, where R⁵ represents H or an alkyl group having 1 to 4 carbon atoms, “b” represents an integer of from 1 to 500, “c” represents an integer of from 1 to 100, and repeating structures represented by “b” and “c” may include random sequences.

### <Method of Producing Compound Represented by Formula (6B)>

The compound represented by the formula (6B) is a known compound and may be synthesized by various methods, and hence is not particularly limited. For example, there is given the following method.

The compound represented by the formula (6B) is synthesized by polymerizing a mixture of octamethyltetracyclosiloxane (D4) and 1,3,5,7-tetramethylcyclotetrasiloxane (D′4), for example, through a reaction of an acid catalyst such as trifluoromethanesulfonic acid in the presence of 1,3-bis [3-(meth)acryloxypropyl]tetramethyldisiloxane serving as a terminal block. “b” and “c” in the formula (6B) can be obtained as desired values by controlling the molar ratios of the terminal block, the D4, and the D′4.

### <Hydrosilylation Reaction>

The hydrosilylation reaction is a reaction known to a person skilled in the art and refers to a reaction in which a hydrosilane having a silicon-hydrogen bond is added to an unsaturated bond to generate a silicon-carbon bond in the presence of a metal catalyst.

Examples of the metal catalyst include a rhodium catalyst typified by Wilkinson catalyst, and a platinum catalyst typified by a platinum chloride catalyst and Karstedt catalyst. Of those, a platinum chloride catalyst and Karstedt catalyst are preferred.

### <Reaction Between Epoxy Group and Carboxylic Acid>

The reaction between an epoxy group and a carboxylic acid is a reaction known to a person skilled in the art and refers to a reaction in which a bond represented by the formula (8) is formed along with ring opening of the epoxy group in the presence of a base catalyst. The base catalyst is not particularly limited, but examples thereof include triethylamine, dimethylamine, diisopropylethylamine, diisopropylamine, sodium hydroxide, and potassium hydroxide. Of those, triethylamine and sodium hydroxide are preferred.

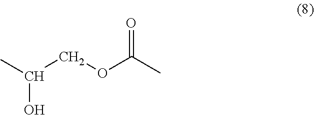

### <Method of Producing Compound Represented by Formula (1B)>

The compound represented by the formula (1B) is synthesized from the compound represented by the formula (6B) by combining a hydrosilylation reaction and a reaction between an epoxy group and a carboxylic acid. A method of producing the compound represented by the formula (1B) is described below. More specifically, the compound by the formula (1B) may be produced by: a hydrosilylation reaction between the compound represented by the formula (6B) and a vinyl compound having an epoxy group, and further a reaction between an epoxy group of a compound after the reaction and a carboxylic acid of a carboxylic acid compound having a phosphorylcholine group; or a reaction between an epoxy group of a vinyl compound having an epoxy group and a carboxylic acid of a carboxylic acid compound having a phosphorylcholine group, and further a hydrosilylation reaction between a vinyl compound having a phosphorylcholine group and a hydroxy group after the reaction and the compound represented by the formula (6B).

The compound represented by the formula (6B) and an excess amount of a vinyl compound having an epoxy group represented by the formula (9) are mixed to perform a hydrosilylation reaction, to thereby provide a compound in which a hydrosilane in the formula (6B) is added to a vinyl group of the formula (9). The unreacted formula (9) compound may be removed by a concentration operation with an evaporator or the like.

Subsequently, a carboxylic acid compound having a phosphorylcholine group represented by the formula (10) is added to perform a reaction between the epoxy group and the carboxylic acid. The reaction between the epoxy group and the carboxylic acid forms the bond represented by the formula (8) to provide a polydimethylsiloxane-containing monomer having a phosphorylcholine group and a hydroxy group represented by the formula (1B). The unreacted carboxylic acid compound having a phosphorylcholine group represented by the formula (10) may be removed by liquid separation purification.

Another example of the method of producing the compound represented by the formula (1B) is the following method.

A vinyl compound having a phosphorylcholine group and a hydroxy group represented by the formula (11) is synthesized by a reaction between the epoxy group of an excess amount of the vinyl compound having an epoxy group represented by the formula (9) and the carboxylic acid of the carboxylic acid compound having a phosphorylcholine group represented by the formula (10). In this case, the unreacted formula (9) compound may be removed by a concentration operation with an evaporator or the like.

Subsequently, the formula (6B) is added to perform a hydrosilylation reaction between the compound represented by the formula (6B) and an excess amount of the vinyl compound having a phosphorylcholine group and a hydroxy group represented by the formula (11). When the hydrosilane in the formula (6B) is added to the vinyl group of the vinyl compound having a phosphorylcholine group and a hydroxy group represented by the formula (11), the polydimethylsiloxane-containing monomer having a phosphorylcholine group and a hydroxy group represented by the formula (1B) is obtained. In this case, the unreacted vinyl compound having a phosphorylcholine group and a hydroxy group represented by the formula (11) may be removed by a liquid separation operation.

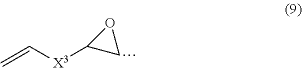

In the formula, X³ represents an alkyl group having 1 to 6 carbon atoms or a divalent group of —R¹²—O—R¹³—, where R¹² represents a divalent alkylene group having 1 to 4 carbon atoms and R¹³ represents a divalent alkylene group having 1 to 6 carbon atoms.

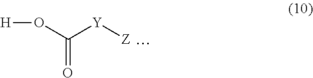

In the formula, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group.

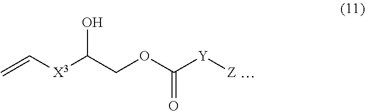

In the formula, X³ represents an alkyl group having 1 to 6 carbon atoms or a divalent group of —R¹²—O—R¹³—, where R¹² represents a divalent alkylene group having 1 to 4 carbon atoms and R¹³ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group.

### <With Regard to Vinyl Compound Having Epoxy Group Represented by Formula (9)>

The vinyl compound having an epoxy group, which is used for synthesis of the formula (1B), is represented by the formula (9).

Examples of the compound of the formula (9) include 3,4-epoxy-1-butene, 1,2-epoxy-5-hexene, allyl glycidyl ether, and 1,2-epoxy-9-decene.

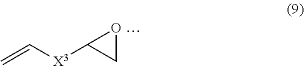

In the formula, X³ represents an alkyl group having 1 to 6 carbon atoms or a divalent group of —R¹²—O—R¹³—, where R¹² represents a divalent alkylene group having 1 to 4 carbon atoms and R¹³ represents a divalent alkylene group having 1 to 6 carbon atoms.

### <With Regard to Carboxylic Acid Compound Having Phosphorylcholine Group Represented by Formula (10)>

The carboxylic acid compound having a phosphorylcholine group, which is used for synthesis of the formula (1B), is represented by the formula (10).

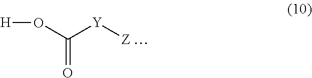

In the formula, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group.

As the carboxylic acid compound having a phosphorylcholine group represented by the formula (10), for example, in JP 2005-187456 A, there is a description of a compound represented by {in the formula (10), Y: —CH₂—, Z: the formula (5)}.

In addition, as the carboxylic acid compound having a phosphorylcholine group represented by the formula (10), for example, in JP 2017-88530 A, there is a description of a compound represented by {in the formula (10), Y: the formula (3), Z: the formula (5)}.

Further, as the carboxylic acid compound having a phosphorylcholine group represented by the formula (10), for example, in JP 2013-234160 A, there is a description of a compound represented by {in the formula (10), Y: the formula (4), Z: the formula (5)}.

### <With Regard to Vinyl Compound Having Phosphorylcholine Group and Hydroxy Group Represented by Formula (11)>

The vinyl compound having a phosphorylcholine group and a hydroxy group represented by the formula (11), which is used for synthesis of the formula (1B), is synthesized by a reaction between the epoxy group of the vinyl compound having an epoxy group represented by the formula (9) and the carboxylic acid of the carboxylic acid compound having a phosphorylcholine group represented by the formula (10).

The vinyl compound having a phosphorylcholine group and a hydroxy group represented by the formula (11) is used for synthesis of the polydimethylsiloxane-containing monomer having a phosphorylcholine group and a hydroxy group represented by the formula (1B) by the hydrosilylation reaction with the compound represented by the formula (6B).

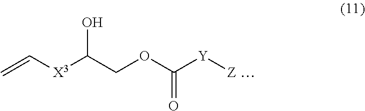

In the formula, X³ represents an alkyl group having 1 to 6 carbon atoms or a divalent group of —R¹²—O—R¹³—, where R¹² represents a divalent alkylene group having 1 to 4 carbon atoms and R¹³ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group.

### <With Regard to Composition of the Present Disclosure>

The composition of the present disclosure includes: at least one kind of polydimethylsiloxane-containing monomer having a phosphorylcholine group and a hydroxy group represented by the formula (1B); at least one kind of hydrophilic monomer; and at least one kind of hydrophilic polymer.

Alternatively, the composition of the present disclosure includes: at least one kind of polydimethylsiloxane-containing monomer having a phosphorylcholine group and a hydroxy group represented by the formula (1B); at least one kind of hydrophilic monomer; at least one kind of hydrophilic polymer; and at least one kind of hydroxy group-containing siloxane monomer.

The composition of the present disclosure may be polymerized through use of a catalyst or an initiator known to a person skilled in the art.

In the composition of the present disclosure, the content of the polydimethylsiloxane-containing monomer having a phosphorylcholine group and a hydroxy group represented by the formula (1B) is from 5 parts by mass to 50 parts by mass, preferably from 10 parts by mass to 40 parts by mass, more preferably from 15 parts by mass to 35 parts by mass with respect to the total amount of the monomers. When the content is less than 5 parts by mass, the transparency of a polymerization product obtained by polymerization is lowered. When the content is more than 50 parts by mass, the surface wettability of the polymerization product is lowered.

In the composition of the present disclosure, the content of the hydrophilic monomer is typically from 20 parts by mass to 90 parts by mass, preferably from 40 parts by mass to 80 parts by mass with respect to the total amount of the monomers.

In the composition of the present disclosure, the content of the hydroxy group-containing siloxane monomer is typically from 20 parts by mass to 90 parts by mass, preferably from 40 parts by mass to 80 parts by mass with respect to the total amount of the monomers.

In the composition of the present disclosure, the content of the hydrophilic polymer is from 0.1 part by mass to 15 parts by mass, preferably from 1 part by mass to 10 parts by mass with respect to the total amount of the composition.

### <With Regard to Hydrophilic Monomer>

The hydrophilic monomer is a compound having a hydrophilic functional group and a polymerizable vinyl group. For example, the hydrophilic monomer may be selected from the group consisting of: 2-hydroxyethyl (meth)acrylate; N-vinylpyrrolidone; N, N-dimethylacrylamide; 2-(methacryloyloxyethyl)-2-(trimethylammonioethyl) phosphate; 2-hydroxypropyl (meth)acrylate; 2-hydroxybutyl (meth)acrylate; N-methyl-N-vinylacetamide; and mixtures thereof, but is not particularly limited thereto.

### <With Regard to Hydrophilic Polymer>

Examples of the hydrophilic polymer may include polyamide, polylactam, polyimide, and polylactone. Those hydrophilic polymers are each preferably a hydrogen bond acceptor that effectively becomes more hydrophilic through a hydrogen bond to water in an aqueous environment.

The hydrophilic polymer is preferably a linear polymer having a cyclic moiety in a polymer main chain thereof. The cyclic moiety is more preferably a cyclic moiety in a cyclic amide or imide. A polymer of this kind preferably includes, for example, polyvinylpyrrolidone and polyvinylimidazole, but a polymer such as polydimethylacrylamide is also useful in the ability thereof. Polyvinylpyrrolidone is the most preferred hydrophilic polymer.

The molecular weight of the hydrophilic polymer is not particularly limited, but is generally from 100,000 to 500,000, more preferably from 300,000 to 500,000.

### <With Regard to Other Components of Composition>

The composition of the present disclosure may include other components except the polydimethylsiloxane-containing monomer having a phosphorylcholine group and a hydroxy group represented by the formula (1B), the hydrophilic monomer, and the hydrophilic polymer.

An example of the other components of the composition of the present disclosure is a siloxane monomer. When the siloxane monomer is added to the composition, the oxygen permeability and dynamic characteristics of a polymerization product of the composition can be improved. The siloxane monomer is not particularly limited as long as the siloxane monomer is a compound containing a siloxane and a vinyl group, but is preferably a hydroxy group-containing siloxane monomer from the viewpoint of compatibility with the composition. The hydroxy group-containing siloxane monomer is a compound containing a siloxane, a vinyl group, and a hydroxy group, and examples thereof include 2-hydroxy-3-[3-[methylbis(trimethylsiloxy) silyl]propoxy]propyl methacrylate and 4-(2-hydroxyethyl)=1-[3-tris(trimethylsiloxy) silylpropyl]=2-methylidene succinate.

The content of the siloxane monomer or the hydroxy group-containing siloxane monomer is typically from 5 parts by mass to 50 parts by mass, preferably from 10 parts by mass to 40 parts by mass, more preferably from 15 parts by mass to 30 parts by mass with respect to the total amount of the monomers.

Another example of the other components of the composition of the present disclosure is a cross-linking agent. The cross-linking agent is known to a person skilled in the art, and for example, is selected from the group consisting of: tetra(ethylene glycol) di(meth)acrylate; tri (ethylene glycol) di(meth)acrylate; ethylene glycol di(meth)acrylate; di(ethylene glycol) di(meth)acrylate; glycerol dimethacrylate; allyl (meth)acrylate; N, N′-methylenebis(meth)acrylamide; N, N′-ethylenebis(meth)acrylamide; N, N′-dihydroxyethylenebis(meth)acrylamide; triallyl isocyanurate; tetraethylene glycol divinyl ether; triethylene glycol divinyl ether; diethylene glycol divinyl ether; ethylene glycol divinyl ether; and combinations thereof. The content of the cross-linking agent is typically from 0.1 part by mass to 5 parts by mass, preferably from 0.3 part by mass to 3 parts by mass, more preferably from 0.5 part by mass to 2 parts by mass with respect to the total amount of the composition.

The combination of the polydimethylsiloxane-containing monomer having a phosphorylcholine group and a hydroxy group represented by the formula (1B), the hydrophilic monomer, and the hydrophilic polymer in the composition of the present disclosure is not particularly limited, but preferred combinations are, for example, as described below.

The polydimethylsiloxane-containing monomer having a phosphorylcholine group and a hydroxy group represented by the formula (1B), N, N-dimethylacrylamide (DMAA), 2-hydroxybutyl methacrylate (HBMA), N-vinylpyrrolidone (NVP), and polyvinylpyrrolidone (PVP)

The polydimethylsiloxane-containing monomer having a phosphorylcholine group and a hydroxy group represented by the formula (1B), DMAA, 2-hydroxypropyl methacrylate (HPMA), NVP, and PVP

The polydimethylsiloxane-containing monomer having a phosphorylcholine group and a hydroxy group represented by the formula (1B), DMAA, HBMA, NVP, N-methyl-N-vinylacetamide (MVA), and PVP

The polydimethylsiloxane-containing monomer having a phosphorylcholine group and a hydroxy group represented by the formula (1B), DMAA, HBMA, NVP, 2-(methacryloyloxyethyl)-2-(trimethylammonioethyl) phosphate (MPC), and PVP The polydimethylsiloxane-containing monomer having a phosphorylcholine group and a hydroxy group represented by the formula (1B), DMAA, 2-hydroxyethyl methacrylate (HEMA), NVP, and PVP

The polydimethylsiloxane-containing monomer having a phosphorylcholine group and a hydroxy group represented by the formula (1B), DMAA, HBMA, NVP, PVP, and 4-(2-hydroxyethyl)=1-[3-tris(trimethylsiloxy) silylpropyl]=2-methylidene succinate (ETS)

The polydimethylsiloxane-containing monomer having a phosphorylcholine group and a hydroxy group represented by the formula (1B), DMAA, HPMA, NVP, PVP, and ETS

The polydimethylsiloxane-containing monomer having a phosphorylcholine group and a hydroxy group represented by the formula (1B), DMAA, HBMA, NVP, MVA, and ETS

The polydimethylsiloxane-containing monomer having a phosphorylcholine group and a hydroxy group represented by the formula (1B), DMAA, HBMA, NVP, MPC, and 2-hydroxy-3-[3-[methylbis(trimethylsiloxy) silyl]propoxy]propyl methacrylate (SiGMA)

The polydimethylsiloxane-containing monomer having a phosphorylcholine group and a hydroxy group represented by the formula (1B), DMAA, HEMA, NVP, PVP, and ETS

### <With Regard to Polymerization Product of the Present Disclosure>

A polymerization product of the present disclosure may be obtained by polymerizing the composition of the present disclosure, and in the polymerization, any solvent and any thermal initiator or photoinitiator serving as a radical initiator for polymerization may be used.

Further, a polymerization method is not particularly limited, and various methods well-known to a person skilled in the art may be used. For example, the polymerization may be performed by a known method including mixing and uniformly dissolving the composition, appropriately adding a thermal polymerization initiator or a photopolymerization initiator typified by a peroxide or an azo compound, and dispensing the resultant into a contact lens mold, followed by heating, UV irradiation, or the like. The polymerization may be performed in the atmosphere, but may be performed under an atmosphere of an inert gas, such as nitrogen or argon, for the purpose of improving a polymerization rate. When the polymerization is performed under an atmosphere of an inert gas, the pressure in a polymerization system is desirably set to 1 kgf/cm² or less.

Suitable examples of the solvent include, but not limited to, methanol, ethanol, 1-propanol, 2-propanol, 1-butanol, 2-butanol, tert-butanol, 1-pentanol, 2-pentanol, tert-amyl alcohol, 1-hexanol, 1-octanol, 1-decanol, 1-dodecanol, glycolic acid, lactic acid, and acetic acid. Those solvents may be used alone or as a mixture thereof. One or more kinds selected from ethanol, 1-propanol, 2-propanol, and 1-hexanol are preferred from the viewpoints of availability and pH stability.

A suitable thermal polymerization initiator is known to a person skilled in the art, and examples thereof include a peroxide, a hydroperoxide, an azo-bis(alkyl- or cycloalkylnitrile), a persulfate, a percarbonate, and mixtures thereof. Examples thereof include benzoyl peroxide, tert-butyl peroxide, di-tert-butyl-diperoxyphthalate, tert-butyl hydroperoxide, azo-bis(isobutyronitrile) (AIBN), 1,1-azodiisobutylamidine, 1,1′-azo-bis(1-cyclohexanecarbonitrile), and 2,2′-azo-bis(2,4-dimethylvaleronitrile). The polymerization is conveniently performed at a high temperature, such as a temperature of from 25° C. to 140° C., preferably from 40° C. to 120° C. in the above-mentioned solvent. A reaction time may vary within a wide limit, but is conveniently, for example, from 1 hour to 24 hours or preferably from 1.5 hours to 12 hours. It is advantageous that the components and the solvent to be used in the polymerization reaction be degassed in advance and the above-mentioned copolymerization reaction be performed under an inert atmosphere, for example, under a nitrogen or argon atmosphere.

Suitable examples of the photoinitiator include benzoin methyl ether, diethoxyacetophenone, benzoylphosphine oxide, and 1-hydroxycyclohexyl phenyl ketone. Of those, Darocur (registered trademark) 1173 and Darocur (registered trademark) 2959, Irgacure (registered trademark) 819, and a germanium-based Norrish-type I photoinitiator are preferred. Examples of the benzoylphosphine oxide initiator include 2,4,6-trimethylbenzoyldiphenylphosphine oxide, bis-(2,6-dichlorobenzoyl)-4-N-propylphenylphosphine oxide, and bis-(2,6-dichlorobenzoyl)-4-N-butylphenylphosphine oxide. For example, a reactive photoinitiator that may be incorporated into a macromer or may be used as a special monomer is also suitable.

After the polymerization, the polymerization product may be released from the mold by a known method and taken out in a dry state. Alternatively, the polymerization product may also be released by immersing the polymerization product in a solvent (e.g., water, ethanol, 1-propanol, 2-propanol, and a mixed solution thereof) together with the mold to swell the polymerization product. Further, the resultant may be washed by being repeatedly immersed in those solvents to remove residues of each of components, residual materials, by-products, and the like, to thereby provide a polymerization product.

Examples of the solvent to be used for washing include water, ethanol, 1-propanol, 2-propanol, and mixtures thereof. For washing, for example, the polymerization product may be immersed in an alcohol-based solvent at a temperature of from 10° C. to 40° C. for from 10 minutes to 10 hours. In addition, after the washing with an alcohol-based solvent, the resultant may be washed by being immersed in an aqueous solution having an alcohol concentration of from 20 mass % to 50 mass % for from 10 minutes to 10 hours.

Any solvent may be used as the solvent to be used for washing as long as the solvent has quality for drugs, quasi-drugs, and medical devices.

### <With Regard to Ophthalmic Device of the Present Disclosure>

An ophthalmic device of the present disclosure is substantially formed of the polymerization product of the present disclosure or includes the polymerization product of the present disclosure. The ophthalmic device in the present disclosure encompasses a contact lens, a soft contact lens, a hard contact lens, an intraocular lens, and an artificial cornea, but is not particularly limited thereto.

### Configuration Examples of Compound Represented by Formula (1B) of the Present Disclosure

Configuration examples of the compound represented by the formula (1B) of the present disclosure are shown in Table 1 below, but are not particularly limited thereto.

In addition, R², W², “b”, “c”, and X¹, Y, and Z of A⁰ shown in Table 1 may be each changed to other combinations to form the compound represented by the formula (1B) of the present disclosure.

### EXAMPLES

The present disclosure is described in more detail below by way of Examples and Comparative Examples. However, the present disclosure is not limited thereto.

Conditions for ¹H NMR analysis performed in Examples are shown below.
Measurement apparatus: JNM-AL400 manufactured by JEOL Ltd.
Solvent: DMSO-d6
Relaxation time: 15 seconds
Number of scans: 32 times

### <Synthesis of Polydimethylsiloxane-Containing Monomer Having Phosphorylcholine Group and Hydroxy Group>

### [Synthesis Example 1-1] Synthesis of Compound Represented by Formula (6B)

8.70 g (10.2 mmol) of X-22-164AS (manufactured by Shin-Etsu Chemical Co., Ltd., dual-end methacryloyloxypropyl polydimethylsiloxane (molecular weight≈1,000)), 20.3 g (68.3 mmol) of octamethyltetracyclosiloxane (D4), and 1.76 g (7.33 mmol) of 1,3,5,7-tetramethylcyclotetrasiloxane (D′4) were mixed in a 100 mL light-shielding bottle, and further 0.05 g of trifluoromethanesulfone was added. After the mixture was subjected to a reaction at 25° C. for 6 hours, 0.26 g of sodium hydrogencarbonate was added to terminate the reaction by neutralization. Subsequently, 3.07 g of sodium sulfate was added to perform dehydration for 30 minutes. Sodium hydrogencarbonate and sodium sulfate were removed by pressure filtration. Thus, 28.35 g of the formula (6B) {R²: —CH₃, W²: —O—, b: 34, c: 3} was obtained.

### [Synthesis Examples 1-2 to 1-7] Synthesis of Compound Represented by Formula (6B)

Formula (6B) compounds of Synthesis Examples 1-2 to 1-7 were each obtained as described below by the same procedure as that in Synthesis Example 1-1 except that the loading amounts of the X-22-164AS, the D4, and the D′4 were changed.
Synthesis Example 1-2: the formula (6B) {R²: —CH₃, W²: —O—, b: 36, c: 2}
Synthesis Example 1-3: the formula (6B) {R²: —CH₃, W²: —O—, b: 35, c: 1}
Synthesis Example 1-4: the formula (6B) {R²: —CH₃, W²: —O—, b: 34, c: 3}
Synthesis Example 1-5: the formula (6B) {R²: —CH₃, W²: —O—, b: 70, c: 5}
Synthesis Example 1-6: the formula (6B) {R²: —CH₃, W²: —O—, b: 90, c: 12}
Synthesis Example 1-7: the formula (6B) {R²: —CH₃, W²: —O—, b: 141, c: 20}
Synthesis Example 1-8: the formula (6B) {R²: —CH₃, W²: —O—, b: 275, c: 69}

### [Example 1-1] Synthesis of Compound Represented by Formula (1B)

After 3.00 g of the compound represented by the formula (6B) obtained in Synthesis Example 1-1 and 0.49 g of allyl glycidyl ether were dissolved in 6.48 g of toluene, 15 μL of Karstedt catalyst (manufactured by Tokyo Chemical Industry Co., Ltd.) adjusted to a 10 wt % xylene solution was added to perform a hydrosilylation reaction overnight. 0.01 g of activated carbon was added, and the mixture was stirred for 30 minutes. The activated carbon was removed by pressure filtration, and the filtrate was concentrated with an evaporator.

Subsequently, 1.29 g of the formula (10) {in the formula (10), Y: the formula (3), Z: the formula (5)} compound of this description, which is described also in JP 2017-88530 A, was added. Then, 4.88 g of methanol, 4.88 g of 2-propanol, and 0.13 g of triethylamine were added to perform a reaction (reaction between an epoxy group and a carboxylic acid) overnight in a reflux state.

After the reaction, an insoluble portion was removed by pressure filtration, and the filtrate was concentrated with an evaporator. The residue was mixed with 15.36 g of ion-exchanged water, 5.12 g of 2-propanol, and 15.36 g of heptane, followed by stirring. After being left to stand still, the mixture was separated into two layers, and the lower layer was discarded. After that, the mixing with ion-exchanged water, 2-propanol, and heptane, the stirring, and the discarding of the lower layer were repeated twice, and the remaining layer was concentrated with an evaporator to provide 2.95 g of a viscous material.

It was recognized from ¹H NMR analysis that a compound was represented by the formula (1B) {R²: —CH₃, W²: —O—, b: 34, c: 3, in A⁰, X¹: —CH₂CH₂CH₂—O—CH₂—, Y: the formula (3), Z: the formula (5)} was obtained. The synthesis results of Example 1-1 are shown in Table 1.

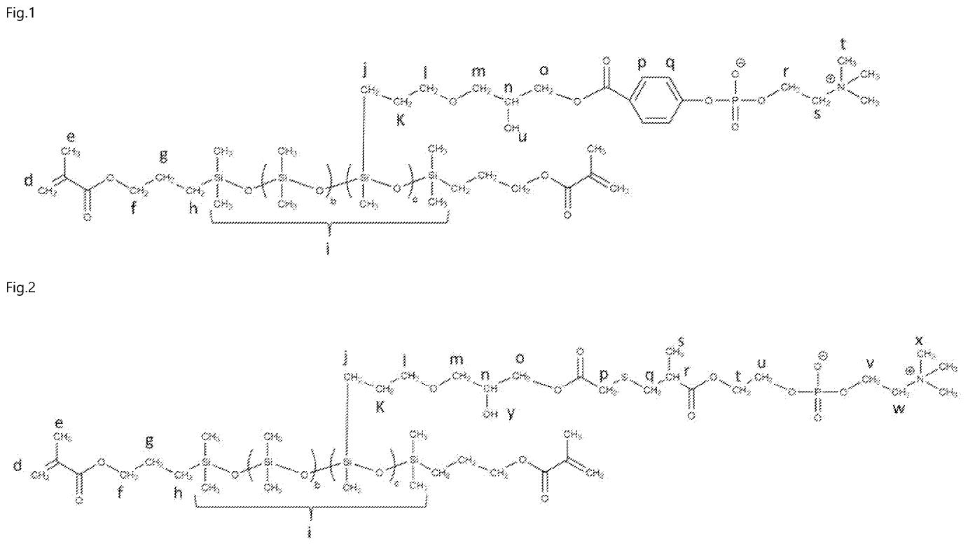

The attribution results of the ¹H NMR analysis of Example 1-1 are shown below (FIG. 1).

¹H NMR (DMSO-d6): δ=0.0 ppm (i), 0.6 ppm (h, j), 1.5 ppm to 1.8 ppm (g, k), 1.9 ppm (e), 3.2 ppm (t), 3.3 ppm to 3.8 ppm (1, m, o, s), 3.8 ppm to 4.5 ppm (f, n, r), 5.6 ppm, 6.1 ppm (d), 5.8 ppm (u), 7.3 ppm (q), 8.0 ppm (p)

### [Example 1-2] Synthesis of Compound Represented by Formula (1B)

After 1.24 g of the formula (10) {in the formula (10), Y: the formula (4), R³: —CH₃, X²: —CH₃, Z: the formula (5)} compound of this description, which is described also in JP 2013-234160 A, 0.41 g of allyl glycidyl ether, 1.53 g of methanol, 1.53 g of 2-propanol, and 0.098 g of triethylamine were added, the temperature of the mixture was raised to perform a reaction overnight in a reflux state. After the reaction, an insoluble portion was removed by pressure filtration, and the filtrate was concentrated with an evaporator.

Subsequently, 3.00 g of the compound represented by the formula (6B) obtained in Synthesis Example 1-2, 5.02 g of 2-propanol, and 50 μL of a platinum chloride catalyst adjusted to a 4 wt % 2-propanol solution were added to perform a reaction overnight. 0.05 g of activated carbon was added, and the mixture was stirred for 30 minutes. The activated carbon was removed by pressure filtration, and the filtrate was concentrated with an evaporator.

The residue was mixed with 17.1 g of ion-exchanged water, 5.70 g of 2-propanol, and 17.1 g of heptane, followed by stirring. After being left to stand still, the mixture was separated into two layers, and the lower layer was discarded. After that, the mixing with ion-exchanged water, 2-propanol, and heptane, the stirring, and the discarding of the lower layer were repeated twice, and the remaining layer was concentrated with an evaporator to provide 2.23 g of a viscous material.

It was recognized from ¹H NMR analysis that a compound represented by the formula (1B) {R²: —CH₃, W²: —O—, b: 36, c: 2; in A⁰, X¹: —CH₂CH₂CH₂—O—CH₂—, Y: the formula (4), R³: —CH₃, X²:—CH₃, Z: the formula (5)} was obtained. The synthesis results of Example 1-2 are shown in Table 1.

The attribution results of the ¹H NMR analysis of Example 1-2 are shown below (FIG. 2).

¹H NMR (DMSO-d6): δ=0.0 ppm (i), 0.5 ppm to 0.6 ppm (h, j), 1.2 ppm(s), 1.6 ppm to 1.7 ppm (g, k), 1.9 ppm (e), 2.5 ppm to 2.9 ppm (q, r), 3.2 ppm (x), 3.3 ppm to 3.8 ppm (1, m, o, p, w), 3.9 ppm to 4.7 ppm (f, n, t, u, v), 5.6 ppm, 6.1 ppm (d), 5.8 ppm (y)

### [Example 1-3] Synthesis of Compound Represented by Formula (1B)

The formula (1B) {R²: —CH₃, W²: —O—, b: 35, c: 1; in A⁰, X¹; —CH₂CH₂CH₂—O—CH₂—, Y: —CH₂—, Z: the formula (5)} was obtained by performing a reaction through use of the formula (6B) compound obtained in Synthesis Example 1-3 and through use of the formula (10) {Y: —CH₂—, Z: the formula (5)} compound, which is described in JP 2005-187456 A, as the carboxylic acid compound having a phosphorylcholine group represented by the formula (10) by the same method as that in Example 1-1. The synthesis results of Example 1-3 are shown in Table 1.

### [Example 1-4] Synthesis of Compound Represented by Formula (1B)

A reaction was similarly performed through use of the formula (6B) compound obtained in Synthesis Example 1-4 and through use of 1,2-epoxy-5-hexene instead of allyl glycidyl ether by the same method as that in Example 1-1 to provide the formula (1B) {R²: —CH₃, W²: —O—, b: 34, c: 3; in A⁰, X¹:—CH₂CH₂CH₂CH₂—, Y: the formula (3), Z: the formula (5)}. The synthesis results of Example 1-4 are shown in Table 1.

### [Example 1-5] Synthesis of Compound Represented by Formula (1B)

A reaction was similarly performed through use of the formula (6B) compound obtained in Synthesis Example 1-5 by the same method as that in Example 1-1 to provide the formula (1B) {R²: —CH₃, W²: —O—, b: 70, c: 5; in A⁰, X¹: —CH₂CH₂CH₂—O—CH₂—, Y: the formula (3), Z: the formula (5)}. The synthesis results of Example 1-5 are shown in Table 1.

### [Example 1-6] Synthesis of Compound Represented by Formula (1B)

A reaction was similarly performed through use of the formula (6B) compound obtained in Synthesis Example 1-6 by the same method as that in Example 1-1 to provide the formula (1B) {R²: —CH₃, W²: —O—, b: 90, c: 12; in A⁰, X¹: —CH₂CH₂CH₂—O—CH₂—, Y: the formula (3), Z: the formula (5)}. The synthesis results of Example 1-6 are shown in Table 1.

### [Example 1-7] Synthesis of Compound Represented by Formula (1B)

A reaction was similarly performed through use of the formula (6B) compound obtained in Synthesis Example 1-7 by the same method as that in Example 1-1 to provide the formula (1B) {R²: —CH₃, W²: —O—, b: 141, c: 20; in A⁰, X¹: —CH₂CH₂CH₂—O—CH₂—, Y: the formula (3), Z: the formula (5)}. The synthesis results of Example 1-7 are shown in Table 1.

### [Example 1-8] Synthesis of Compound Represented by Formula (1B)

A reaction was similarly performed through use of the formula (6B) compound obtained in Synthesis Example 1-8 by the same method as that in Example 1-1 to provide the formula (1B) {R²: —CH₃, W²: —O—, b: 275, c: 69; in A⁰, X¹: —CH₂CH₂CH₂—O—CH₂—, Y: the formula (3), Z: the formula (5)}. The synthesis results of Example 1-8 are shown in Table 1.

### [Synthesis Example 1-9] Synthesis of Formula (1B) Comparative Compound

8.40 g of X-22-164AS (manufactured by Shin-Etsu Chemical Co., Ltd., dual-end methacryloyloxypropyl polydimethylsiloxane (molecular weight≈1,000)) and 21.43 g of D4 were mixed, and further 0.04 g of trifluoromethanesulfonic acid was added. After the mixture was subjected to a reaction at 25° C. for 6 hours, 0.25 g of sodium hydrogencarbonate was added to terminate the reaction by neutralization. Subsequently, 2.97 g of sodium sulfate was added to perform dehydration for 30 minutes. Sodium hydrogencarbonate and sodium sulfate were removed by pressure filtration. Thus, 28.6 g of a formula (1B) comparative compound that was dual-end methacryloyloxypropyl polydimethylsiloxane {corresponding to R²: —CH₃, W²: —O—, b: 37 in the formula (1B)} was obtained. The synthesis results of Synthesis Example 1-9 are shown in Table 1.

### [Synthesis Example 1-10] Synthesis of Formula (1B) Comparative Compound

After 3.00 g of the compound represented by the formula (6B) obtained in Synthesis Example 1-1 and 0.31 g of 3-buten-1-ol were dissolved in 6.45 g of toluene, 15 μL of Karstedt catalyst (manufactured by Tokyo Chemical Industry Co., Ltd.) adjusted to a 10 wt % xylene solution was added to perform a reaction overnight. 0.1 g of activated carbon was added, and the mixture was stirred for 30 minutes. The activated carbon was removed by pressure filtration. Thus, 3.12 g of a formula (1B) comparative compound, which corresponded to R²: —CH₃, W²: —O—, b: 34, c: 3 in the formula (1B) and in which an A⁰ portion was substituted by —CH₂CH₂CH₂CH₂OH, was obtained. The synthesis results of Synthesis Example 1-10 are shown in Table 1.

TABLE 1

| | | Example 1-1 | Example 1-2 | Example 1-3 | Example 1-4 |
|---|---|---|---|---|---|
| R² | | —CH³ | —CH³ | —CH³ | —CH³ |
| W² | | —O— | —O— | —O— | —O— |
| b | | 34 | 36 | 35 | 34 |
| c | | 3 | 2 | 1 | 3 |
| A⁰ | X¹ | —CH₂CH₂CH₂—O—CH₂— | —CH₂CH₂CH₂—O—CH₂— | —CH₂CH₂CH₂—O—CH₂— | —CH₂CH₂CH₂—O—CH₂— |
| | Y | Formula (3) | Formula (4) | —CH₂— | Formula (3) |
| | | | R₃: —CH₃, X₂: —CH₃ | | |
| | Z | Formula (5) | Formula (5) | Formula (5) | Formula (5) |
| | | Example 1-5 | Example 1-6 | Example 1-7 | Example 1-8 |
| R² | | —CH³ | —CH³ | —CH³ | —CH³ |
| W² | | —O— | —O— | —O— | —O— |
| b | | 70 | 90 | 141 | 275 |
| c | | 5 | 12 | 20 | 69 |
| A⁰ | X¹ | —CH₂CH₂CH₂—O—CH₂— | —CH₂CH₂CH₂—O—CH₂— | —CH₂CH₂CH₂—O—CH₂— | —CH₂CH₂CH₂—O—CH₂— |
| | Y | Formula (3) | Formula (3) | Formula (3) | Formula (3) |
| | Z | Formula (5) | Formula (5) | Formula (5) | Formula (5) |

| Formula (1B) | | |
|---|---|---|
| comparative | Synthesis | Synthesis |
| compound | Example 1-9 | Example 1-10 |
| R² | —CH₃ | —CH₃ |
| W² | —O— | —O— |
| b | 37 | 34 |
| c | | 3 |
| Portion | | —CH₂CH₂CH₂OH |
| corresponding | | |
| to A° | | |

### <Polymerization of Composition>

### Components Used in Example 2 and Comparative Example 2

The components of compositions used in Examples 2 and Comparative Example except the polydimethylsiloxane-containing monomer having a phosphorylcholine group and a hydroxy group and the comparative compound thereof are described below.

### Hydrophilic Monomer

DMAA: N, N-dimethylacrylamide
HEMA: 2-hydroxyethyl methacrylate
HPMA: 2-hydroxypropyl methacrylate (mixture of 2-hydroxypropyl ester and 2-hydroxy-1-methylethyl ester)
HBMA: 2-hydroxybutyl methacrylate
NVP: N-vinylpyrrolidone
MVA: N-methyl-N-vinylacetamide
MPC: 2-(methacryloyloxyethyl)-2-(trimethylammonioethyl) phosphate

### Hydroxy Group-Containing Siloxane Monomer

ETS: 4-(2-hydroxyethyl)=1-[3-tris(trimethylsiloxy) silylpropyl]=2-methylidene succinate
SiGMA: 2-hydroxy-3-[3-[methylbis(trimethylsiloxy) silyl]propoxy]propyl methacrylate

### Hydrophilic Polymer

PVP K90: polyvinylpyrrolidone K90 (manufactured by Wako Pure Chemical Industries, Ltd.)

### Cross-Linking Agent

TEGDMA: tetraethylene glycol dimethacrylate

### Solvent

HexOH: 1-hexanol

### Initiator

AIBN: 2,2′-azobis(isobutyronitrile)

### Evaluation Method

Evaluation methods in Examples 2 and Comparative Examples 2 are as described below.

### [Evaluation of Composition Uniformity]

The uniformity of a composition before polymerization was evaluated by the following method.

The prepared composition was placed in a colorless and transparent container, and was visually evaluated by giving a score in accordance with the following criteria.

1: The composition is uniform and transparent.

2: The composition is non-uniform, or white turbidity, a precipitate, or the like is observed.

The composition given the score of “1” was subjected to a polymerization step, and the resultant polymerization product was evaluated for its polymerization product transparency and anti-lipid adhesion property. The composition given the score of “2” was not subjected to the subsequent polymerization step and evaluations for the polymerization product transparency and the anti-lipid adhesion property.

### [Evaluation of Polymerization Product Transparency]

The transparency of a polymerization product obtained by polymerizing a composition was evaluated by the following method.

The polymerization product was immersed in physiological saline overnight, and in a windless room, the polymerization product was taken out from the physiological saline. The polymerization product was exposed to illumination, was visually observed for its appearance, and was evaluated by giving a score in accordance with the following criteria.

1: The polymerization product is transparent without turbidity.

2: The polymerization product is slightly white-turbid.

3: The polymerization product is somewhat white-turbid and semi-transparent.

4: The polymerization product is white-turbid and is not transparent at all.

5: The polymerization product is completely white.

### [Evaluation of Anti-Lipid Adhesion Property]

The anti-lipid adhesion property of a polymerization product was evaluated by the following procedure.

First, artificial lipid was prepared by the method described below. Subsequently, after the polymerization product immersed overnight in physiological saline was immersed in 4 mL of the artificial lipid for 4 hours, the polymerization product was rinsed lightly with physiological saline, and moisture was removed. The polymerization product was visually observed for its appearance, and was evaluated by giving a score in accordance with the following criteria.

1: The polymerization product is transparent without white turbidity.

2: The polymerization product is slightly white-turbid.

3: The polymerization product is partially white-turbid.

4: The polymerization product is mostly white-turbid.

5: The polymerization product is white-turbid in its entirety.

### Preparation of Artificial Lipid

1) 0.5 g of mixed lipid having composition described below was mixed with 100 mL of a phosphate-borate buffer solution described below.

2) The mixture was suspended with a homomixer at 60° C.

3) The mixture was adjusted to a pH of 7.0 with 1 N hydrochloric acid.

Composition of Mixed Lipid

| | |
|---|---|
| Oleic acid | 0.06 g |
| Linolenic acid | 0.06 g |
| Palmitic acid | 0.06 g |
| Tripalmitic acid | 0.81 g |
| Cetyl alcohol | 0.20 g |
| Cetyl myristate | 0.81 g |
| Cholesterol | 0.08 g |
| Cholesterol palmitate | 0.08 g |
| Lecithin (derived from egg) | 2.83 g |

Composition of Phosphate-Borate Buffer Solution

| | |
|---|---|
| Sodium chloride | 2.25 g |
| Potassium dihydrogen phosphate | 1.25 g |
| Sodium tetraborate decahydrate | 5.65 g |
| Ion-exchanged water | Total amount was |
| | set to 250 mL |

Reference: Kaoru Iwai; Mari Moriyama; Masaki Imayasu; Hidenari Tanaka: Lipid Adsorption to Contact Lens Materials., Journal of Japan Contact Lens Society, 1995, 37, 58-61.

### Example 2-1

20.0 Parts by mass of the formula (1B) compound obtained in Example 1-1, 10.0 parts by mass of DMAA, 10.0 parts by mass of HBMA, 30.0 parts by mass of NVP, 30.0 parts by mass of SiGMA, 5.00 parts by mass of PVP K90, 1.00 part by mass of TEGDMA, and 30.0 parts by mass of HexOH were mixed. Subsequently, 0.50 part by mass of AIBN was added to the mixture, followed by mixing. Thus, a composition was produced.

The resultant composition was evaluated for its composition uniformity, and was given the score of “1”.

Subsequently, the composition was dispensed into a contact lens mold, and the mold was placed in an oven. After the inside of the oven was purged with nitrogen, the temperature in the oven was raised to 100° C., and the inside of the oven was maintained at this temperature for 2 hours so that the composition was polymerized. Thus, a polymerization product was obtained.

The polymerization product was taken out from the mold, and was purified by being immersed in 40 g of 2-propanol for 4 hours and then immersed in 50 g of ion-exchanged water for 4 hours so that an unreacted substance and the like were removed. Further, the polymerization product was immersed in physiological saline described in ISO-18369-3 to provide a contact lens-shaped polymerization product.

When the contact lens-shaped polymerization product was evaluated for its polymerization product transparency, the polymerization product was given the score of “1”. When the contact lens-shaped polymerization product was evaluated for its anti-lipid adhesion property, the polymerization product was given the score of “1”. The results of Example 2-1 are shown in Table 2.

### Examples 2-2 to 2-8

Examples 2-2 to 2-8 were each performed in the same manner as in Example 2-1 except that the composition shown in Table 2 was followed. The evaluations of composition uniformity, polymerization product transparency, and an anti-lipid adhesion property were performed in the same manner as in Example 2-1.

The evaluation results of Examples 2-2 to 2-6 are shown in Table 2.

### Comparative Example 2-1

A composition was produced in the same manner as in Example 2-1 except that 20.0 parts by mass of the formula (1B) comparative compound synthesized in Synthesis Example 1-9 was used instead of 20.0 parts by mass of the compound represented by the formula (1B) in accordance with the composition shown in Table 2.

The resultant composition was evaluated for its composition uniformity, and was given the score of “2”. Thus, the composition was not subjected to the subsequent polymerization step and evaluations for the polymerization product transparency and the anti-lipid adhesion property. The evaluation results of Comparative Example 2-1 are shown in Table 2.

### Comparative Example 2-2

Comparative Example 2-2 was performed in the same manner as in Example 2-1 except that 20.0 parts by mass of the formula (1B) comparative compound synthesized in Synthesis Example 1-10 was used instead of 20.0 parts by mass of the compound represented by the formula (1B) in accordance with the composition shown in Table 2. The evaluations of composition uniformity, polymerization product transparency, and an anti-lipid adhesion property were performed in the same manner as in Example 2-1. As a result, the score of “1” was given for the composition uniformity, the score of “4” was given for the polymerization product transparency, and the score of “5” was given for the anti-lipid adhesion property. The evaluation results of Comparative Example 2-2 are shown in Table 2.

TABLE 2

| | | | Example | Example | Example | Example | Example | Example |
|---|---|---|---|---|---|---|---|---|
| | | | 2-1 | 2-2 | 2-3 | 2-4 | 2-5 | 2-6 |
| | | Raw | Mixing | Mixing | Mixing | Mixing | Mixing | Mixing |
| Kind | | material | ratio | ratio | ratio | ratio | ratio | ratio |
| Formula | Part(s) | Example | 20.0 | | | | | |
| (1B) | by | 1-1 | | | | | | |
| compound | mass | Example | | 20.0 | | | | |
| | | 1-2 | | | | | | |
| | | Example | | | 20.0 | | | |
| | | 1-3 | | | | | | |
| | | Example | | | | 20.0 | | |
| | | 1-4 | | | | | | |
| | | Example | | | | | 20.0 | |
| | | 1-5 | | | | | | |
| | | Example | | | | | | 20.0 |
| | | 1-6 | | | | | | |
| | | Example | | | | | | |
| | | 1-7 | | | | | | |
| | | Example | | | | | | |
| | | 1-8 | | | | | | |
| Formula | | Synthesis | | | | | | |
| (1B) | | Example | | | | | | |
| comparative | | 1-9 | | | | | | |
| compound | | Synthesis | | | | | | |
| | | Example | | | | | | |
| | | 1-10 | | | | | | |
| Hydrophilic | | DMAA | 10.0 | 10.0 | 10.0 | 10.0 | 10.0 | 10.0 |
| monomer | | HEMA | | | | | | |
| | | HPMA | | 10.0 | | | 10.0 | |
| | | HBMA | 10.0 | | 10.0 | 10.0 | | 10.0 |
| | | NVP | 30.0 | 30.0 | 20.0 | 20.0 | 30.0 | 30.0 |
| | | MVA | | | 10.0 | | | |
| | | MPC | | | | 10.0 | | |
| Hydroxy | | ETS | | | | 30.0 | | |
| group- | | SiGMA | 30.0 | 30.0 | 30.0 | | 30.0 | 30.0 |
| containing | | | | | | | | |
| siloxane | | | | | | | | |
| monomer | | | | | | | | |
| Total | | — | 100.0 | 100.0 | 100.0 | 100.0 | 100.0 | 100.0 |
| of | | | | | | | | |
| monomers | | | | | | | | |
| Hydro | | PVP K90 | 5.00 | 5.00 | 5.00 | 5.00 | 5.00 | 5.00 |
| philic | | | | | | | | |
| polymer | | | | | | | | |
| Crosslinking | | TEGDMA | 1.00 | 1.00 | 1.00 | 1.00 | 1.00 | 1.00 |
| agent | | | | | | | | |
| Solvent | | HexOH | 30.0 | 30.0 | 30.0 | 30.0 | 30.0 | 30.0 |
| Initiator | | AIBN | 0.50 | 0.50 | 0.50 | 0.50 | 0.50 | 0.50 |
| Polymerization | | Temperature | 100° C. | 100° C. | 100° C. | 100° C. | 100° C. | 100° C. |
| step | | Time | 2 hours | 2 hours | 2 hours | 2 hours | 2 hours | 2 hours |

| Evaluation items | Evaluation results | | | | | |
|---|---|---|---|---|---|---|
| Composition uniformity | 1 | 1 | 1 | 1 | 1 | 1 |
| Polymerization product | 7 | 1 | 7 | 1 | 1 | 2 |
| transparency | | | | | | |
| Anti-lipid adhesion | 1 | 1 | 1 | 1 | 1 | 2 |
| property | | | | | | |

| | | | Example | Example | Comparative | Comparative |
|---|---|---|---|---|---|---|
| | | | 2-7 | 2-8 | Example 2-1 | Example 2-2 |
| | | Raw | Mixing | Mixing | Mixing | Mixing |
| Kind | | material | ratio | ratio | ratio | ratio |
| Formula | Part(s) | Example | | | | |
| (1B) | by | 1-1 | | | | |
| compound | mass | Example | | | | |
| | | 1-2 | | | | |
| | | Example | | | | |
| | | 1-3 | | | | |
| | | Example | | | | |
| | | 1-4 | | | | |
| | | Example | | | | |
| | | 1-5 | | | | |
| | | Example | | | | |
| | | 1-6 | | | | |
| | | Example | 20.0 | | | |
| | | 1-7 | | | | |
| | | Example | | 20.0 | | |
| | | 1-8 | | | | |
| Formula | | Synthesis | | | 20.0 | |
| (1B) | | Example | | | | |
| comparative | | 1-9 | | | | 20.0 |
| compound | | Synthesis | | | | |
| | | Example | | | | |
| | | 1-10 | | | | |
| Hydrophilic | | DMAA | 10.0 | 10.0 | 10.0 | 10.0 |
| monomer | | HEMA | 10.0 | | | |
| | | HPMA | | | | |
| | | HBMA | | 10.0 | 10.0 | 10.0 |
| | | NVP | 30.0 | 30.0 | 30.0 | 30.0 |
| | | MVA | | | | |
| | | MPC | | | | |
| Hydroxy | | ETS | | | | |
| group- | | SiGMA | 30.0 | 30.0 | 30.0 | 30.0 |
| containing | | | | | | |
| siloxane | | | | | | |
| monomer | | | | | | |
| Total | | — | 100.0 | 100.0 | 100.0 | 100.0 |
| of | | | | | | |
| monomers | | | | | | |
| Hydro | | PVP K90 | 5.00 | 5.00 | 5.00 | 5.00 |
| philic | | | | | | |
| polymer | | | | | | |
| Crosslinking | | TEGDMA | 1.00 | 1.00 | 1.00 | 1.00 |
| agent | | | | | | |
| Solvent | | HexOH | 30.0 | 30.0 | 30.0 | 30.0 |
| Initiator | | AIBN | 0.50 | 0.50 | 0.50 | 0.50 |
| Polymerization | | Temperature | 100° C. | 100° C. | — | 100° C. |
| step | | Time | 2 hours | 2 hours | — | 2 hours |

| Evaluation items | Evaluation results | | | |
|---|---|---|---|---|
| Composition uniformity | 1 | 1 | 2 | 1 |
| Polymerization product | 2 | 3 | — | 4 |
| transparency | | | | |
| Anti-lipid adhesion | 3 | 3 | — | 5 |
| property | | | | |

The evaluation results in Table 2 are described below.

Examples 2-1 to 2-5 were each given the score of “1” for the composition uniformity and the score of “1” for the polymerization product transparency, and hence each exhibited significantly satisfactory compatibility with a hydrophilic monomer and a hydrophilic polymer.

Examples 2-6 and 2-7 were each given the score of “1” for the composition uniformity and the score of “2” for the polymerization product transparency, and hence each exhibited sufficiently satisfactory compatibility with a hydrophilic monomer and a hydrophilic polymer.

Example 2-8 was given the score of “1” for the composition uniformity and the score of “3” for the polymerization product transparency, and hence exhibited sufficiently satisfactory compatibility with a hydrophilic monomer and a hydrophilic polymer.

Examples 2-1 to 2-5 were each given the score of “1” for the anti-lipid adhesion property, and hence each exhibited a significantly high anti-lipid adhesion property.

Example 2-6 was given the score of “2” for the anti-lipid adhesion property, and hence exhibited a high anti-lipid adhesion property.

Examples 2-7 and 2-8 were each given the score of “3” for the anti-lipid adhesion property, and hence each exhibited an anti-lipid adhesion property.

The above-mentioned results revealed that the compound represented by the formula (1B) used in each of Examples 2-1 to 2-8 simultaneously exhibited satisfactory compatibility with a hydrophilic monomer and a hydrophilic polymer and the imparting of an anti-lipid adhesion property to a polymerization product.

Comparative Example 2-1 was given the score of “2” for the composition uniformity, and hence did not exhibit satisfactory compatibility with a hydrophilic monomer and a hydrophilic polymer.

Comparative Example 2-2 was given the score of “1” for the composition uniformity but was given the score of “4” for the polymerization product transparency, and hence did not exhibit satisfactory compatibility with a hydrophilic monomer and a hydrophilic polymer. In addition, Comparative Example 2-2 was given the score of “5” for the anti-lipid adhesion property, and hence did not exhibit an anti-lipid adhesion property.

From the above-mentioned the formula (1B) comparative compound used in each of Comparative Examples 2-1 and 2-2 did not simultaneously exhibit satisfactory compatibility with a hydrophilic monomer and a hydrophilic polymer and an anti-lipid adhesion property.

It was recognized that the polydimethylsiloxane-containing monomer having a phosphorylcholine group and a hydroxy group represented by the formula (1B) exhibited satisfactory compatibility with a hydrophilic monomer and a hydrophilic polymer and exhibited an anti-lipid adhesion property when its composition with the hydrophilic monomer and the hydrophilic polymer was polymerized.

### INDUSTRIAL APPLICABILITY

The present disclosure can provide a polydimethylsiloxane-containing monomer having a phosphorylcholine group and a hydroxy group, the monomer having the following characteristics.

(1) The compatibility with a hydrophilic monomer and a hydrophilic polymer is satisfactory.

(2) A polymerization product obtained by polymerizing a composition containing the monomer of the present disclosure, a hydrophilic monomer, and a hydrophilic polymer has transparency and an anti-lipid adhesion property.

1.-8. (canceled)

9. A compound represented by the formula (1B): 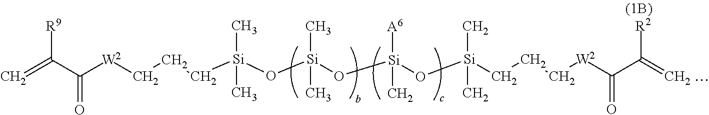
where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2); 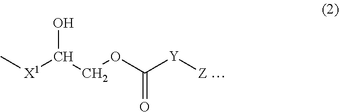
where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group; 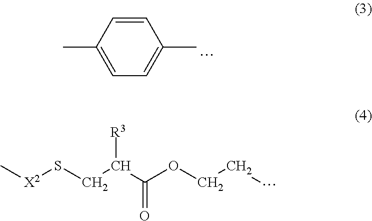
where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms.

10. A method of producing a compound represented by the formula (1B),
the method comprising a step of synthesizing the formula (1B) by:
a hydrosilylation reaction between a compound represented by the formula (6B) and a vinyl compound having an epoxy group, and further a reaction between an epoxy group of a compound after the reaction and a carboxylic acid of a carboxylic acid compound having a phosphorylcholine group; or
a reaction between an epoxy group of a vinyl compound having an epoxy group and a carboxylic acid of a carboxylic acid compound having a phosphorylcholine group, and further a hydrosilylation reaction between a vinyl compound having a phosphorylcholine group and a hydroxy group after the reaction and the compound represented by the formula (6B): 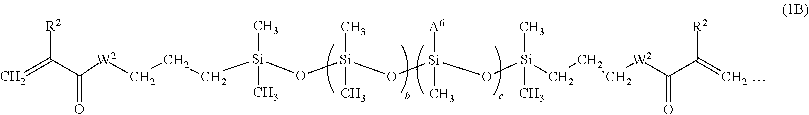
where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2); 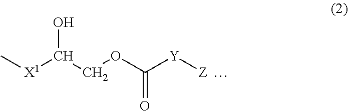
where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group; 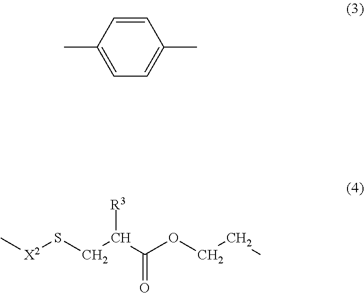
where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms; 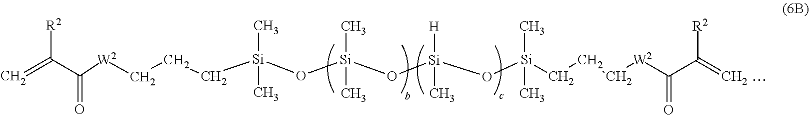
where R² represents H or CH₃, W² represents O or NR⁵, where R⁵ represents H or an alkyl group having 1 to 4 carbon atoms, “b” represents an integer of from 1 to 500, and “c” represents an integer of from 1 to 100.

11. A composition, comprising:
a compound represented by the following formula (1B);
one or more kinds of hydrophilic monomers; and
one or more kinds of hydrophilic polymers: 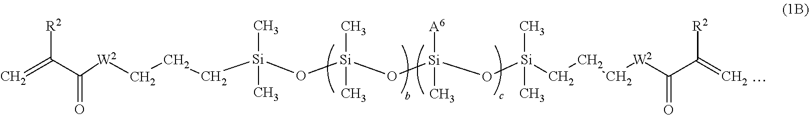
where R² represents H or CH₃, W² represents O or NR⁵, where R⁵ represents H or an alkyl group having 1 to 4 carbon atoms, “b” represents an integer of from 1 to 500, “c” represents an integer of from 1 to 100, and A⁰ is represented by the formula (2); 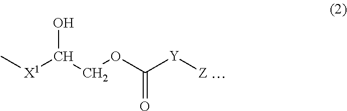
where X¹ represents a divalent alkylene group having 3 to 8 carbon atoms or a divalent group of —R⁶—O—R⁷—, where R⁶ represents a divalent alkylene group having 3 to 6 carbon atoms and R⁷ represents a divalent alkylene group having 1 to 6 carbon atoms, Y represents a divalent alkylene group having 1 to 8 carbon atoms or a divalent group of —R⁸—O—R⁹—, where R⁸ and R⁹ each independently represent a divalent alkylene group having 1 to 6 carbon atoms, or Y is represented by the formula (3) or the formula (4), and Z represents a phosphorylcholine group; 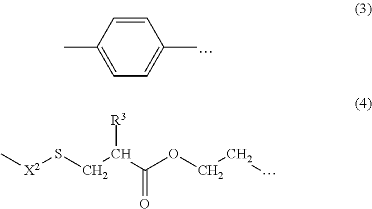
where R³ represents H or CH₃, X² represents a divalent alkylene group having 1 to 10 carbon atoms or a divalent group of —R¹⁰—O—R¹¹—, where R¹⁰ and R¹¹ each independently represent a divalent alkylene group having 1 to 6 carbon atoms.

12. The composition according to claim 11, further comprising one or more kinds of hydroxy group-containing siloxane monomers.

13. The composition according to claim 11, wherein the hydrophilic polymer is one or more selected from the group consisting of: polyamide; polylactam; polyimide; polylactone; and polydextran.

14. The composition according to claim 12, wherein the hydrophilic polymer is one or more selected from the group consisting of: polyamide; polylactam; polyimide; polylactone; and polydextran.

15. The composition according to claim 11, wherein the hydrophilic polymer is polyvinylpyrrolidone.

16. The composition according to claim 12, wherein the hydrophilic polymer is polyvinylpyrrolidone.

17. The compound represented by the formula (1B) according to claim 9, wherein the R² represents CH₃, the W² represents O, X¹ of the A⁰ represents CH₂CH₂CH₂—O—CH₂ or CH₂CH₂CH₂CH₂, and Y of the A⁰ is represented by the formula (3) or the formula (4) (where R³ represents CH₃ and X² represents CH₃) or represents CH₂.

18. The composition according to claim 11, wherein the R² represents CH₃, the W² represents O, X¹ of the A⁰ represents CH₂CH₂CH₂—O—CH₂ or CH₂CH₂CH₂CH₂, and Y of the A⁰ is represented by the formula (3) or the formula (4) (where R³ represents CH₃ and X² represents CH₃) or represents CH₂.

19. The composition according to claim 12, wherein the R² represents CH₃, the W² represents O, X¹ of the A⁰ represents CH₂CH₂CH₂—O—CH₂ or CH₂CH₂CH₂CH₂, and Y of the A⁰ is represented by the formula (3) or the formula (4) (where R³ represents CH₃ and X² represents CH₃) or represents CH₂.

20. A polymerization product obtained by polymerizing the composition of claim 11.

21. A polymerization product obtained by polymerizing the composition of claim 12.

22. A polymerization product obtained by polymerizing the composition of claim 13.

23. A polymerization product obtained by polymerizing the composition of claim 14.

24. An ophthalmic device, comprising the polymerization product of claim 20.

25. An ophthalmic device, comprising the polymerization product of claim 21.

26. An ophthalmic device, comprising the polymerization product of claim 22.

27. An ophthalmic device, comprising the polymerization product of claim 23.
